(19) United States
(12) Patent Application Publication
(10) Pub. No.: US 2004/0126343 A1
(43) Pub. Date: Jul. 1, 2004

(54) Nail drug delivery

(76) Inventor: Solomon David Garshowitz, Downsview (CA)

Correspondence Address: Swabey Ogilvy Renault, Suite 1600, 1981 McGill College Avenue, Montreal, QC, H3A 2Y3, CA

(21) Appl. No.: 10/328,038

(22) Filed: Dec. 26, 2002

(51) Int. Cl. A61K 7/04

(52) U.S. Cl. 424/61

(57) ABSTRACT

The present invention provides a solution, for topical application to a nail. The solution comprises between about 30% to about 85% DMSO by weight of the solution, a medicine, water and at least one of a moisturizing agent for assisting to retain moisture in the nail and prevent the nail from dehydrating, a dispersant for assisting to disperse the components in solution to provide a homogeneous solution when applied and when penetrating the nail, and a solubilizing agent for the medicine.

### FIELD OF THE INVENTION

This invention relates to the use of a pharmaceutical solution comprising dimethyl sulfoxide and more particularly to the use of a pharmaceutical solution comprising dimethyl sulfoxide for improved drug delivery through a nail of a patient.

### BACKGROUND OF THE INVENTION

The use of a pharmaceutical solution comprising dimethyl sulfoxide (DMSO) is known. Such solutions have been used to improve the penetration of a medicine into parts of the body through topical application on the skin. The use of dimethyl sulfoxide in such manner has been described in, for example, U.S. Pat. No. 4,575,515 (Sandborn) issued on Mar. 11, 1986 and U.S. Pat. No. 4,652,557 (Sandhorn) issued on Mar. 24, 1987.

Traditionally topical treatments have only been applied to the skin of a patient. Generally topical treatments have limited effectiveness when used on the nail which is throught to be due to the inability of the treatment to sufficiently penetrate the nail plate. The inability of the treatment to penetrate the nail plate results in a lack of delivery or an insufficient delivery of the therapeutic, contained with the topical treatment, to the target site. Many fungal infections in human nails, such as Onychomycosis, are treated by providing the patient with an oral medication that usually requires months of treatment.

The unique properties of the nail, including its thickness and relatively compact construction, create a barrier to the entry of topically applied agents. These properties are discussed in further detail in Walters K. A. and Flynn, G. L., 1983, Int. J. Cosmetic Sci., 5:231-246.

The human nail plate consists of three layers: the dorsal and intermediate layers derived from the matrix, and the ventral layer from the nail bed. The intermediate layer is three-quarters of the whole nail thickness and consists of soft keratin. The upper or dorsal layer is only a few cell layers thick but consists of hard keratin. The upper layer of the nail is the main barrier for drug diffusion into and through the nail plate. The ventral layer consists of soft hyponychial in which many pathological changes occur, as discussed in Runne U and Orfanos C E. (1981) Curr. Probl. Der. 9:102-149.

Both the human nail and stratum corneum are rich in keratin. However, the two barriers behave differently. The human nail is like a hydrophilic gel membrane rather than a lipophilic partition membrane as is the case with the stratum corneum. The rate of chemical penetration into/through the human nail depends upon the water solubility, and molecular size of the chemical. To increase effective chemical concentration into/through the human nail plate, penetration enhancers, which tend to promote diffusion through the skin's horny layer, have been considered. Walters et al. (1985), J Pharm Pharmacol, 37:771-775, found that DMSO retarded the permeation of methanol and hexanol across the nail plate, and isopropyl alcohol had little influence on penetration of methanol. It therefore appears that these penetration enhancers have little promise as accelerators of nail plate permeability. However, in Franz (1992), Dermatology 184(suppl. 1):18-20, it was reported that pretreatment of nail with DMSO resulted in a large increase in an antifungal agent (amorolfine) penetration. However, the method described involved pre-treating the nail with DMSO and then removing excess DMSO prior to the application of the medication.

It is therefore desirable to provide a pharmaceutical formulation for the improved delivery of a therapeutic through a nail of a patient, without the requirement of pre-treating the nail.

### SUMMARY OF THE INVENTION

The present invention provides the use of a pharmaceutical formulation for the improved delivery of a therapeutic through a nail of a patient.

The present invention further provides a solution, for topical application to a nail of a patient, comprising between about 30% to about 85% DMSO, or analogs thereof, by weight of the solution, a medicine, water and at least one of a moisturizing agent for assisting in the retention of moisture in the nail and preventing the nail from dehydrating, a dispersant for assisting the dispersion of the components in solution to provide a homogeneous solution when applied and when penetrating the nail, and a solubilizing agent for the medicine.

The present invention further provides a carrier solution, for use with a medicine, for topical application to a nail of a patient, comprising between about 30% to about 85% DMSO, or analogs thereof, by weight of the solution, water, and at least one of a moisturizing agent for assisting in the retention of moisture in the nail and preventing the nail from dehydrating, a dispersant for assisting the dispersion of the components in solution to provide a homogeneous solution when applied and when penetrating the nail and a solubilizing agent for the medicine.

The present invention further provides the use of a solution for the topical application of a medicine to a nail in a patient, the solution comprising between about 30% to about 85% DMSO, or analogs thereof, by weight of the solution, a medicine, water and at least one of a moisturizing agent for assisting in the retention of moisture in the nail and prevent the nail from dehydrating, a dispersant for assisting the dispersion of the components in solution to provide a homogeneous solution when applied and when penetrating the nail, and a solubilizing agent for the medicine.

### DETAILED DESCRIPTION OF THE PREFERRED EMBODIMENTS

The present invention provides a solution, for topical application to a nail of a patient. In a preferred embodiment of the present invention the solution, for topical application to the nail, comprises between about 30% to about 85% DMSO, or analogs thereof, by weight of the solution, at least one medicine, water and at least one of a moisturizing agent for assisting the retention of moisture in the nail and to prevent the nail from dehydrating, a dispersant for assisting the dispersion of the components in solution to provide a homogeneous solution when applied and when penetrating the nail, and a solubilizing agent for solubilizing the medicine.

In another embodiment of the present invention the DMSO, or analogs thereof, is present in an amount of between about 60% to about 70% by weight of the solution. In a further embodiment of the present invention the DMSO, or analogs thereof, constitutes about 65% by weight of the solution.

The present invention further provides a carrier solution, for use with a medicine, for topical application to a nail. In a preferred embodiment the solution comprises between about 30% to about 85% DMSO, or analogs thereof, by weight of the solution, a polyalcohol for assisting the retention of moisture in the nail and preventing the nail from dehydrating, a dispersant for assisting in the dispersion of the components in solution to provide a homogeneous solution when applied and when penetrating the nail and water. In an alternative embodiment the carrier solution further comprises a solubilizing agent for solubilizing the medicine.

In the solution and the carrier solution discussed above, it will be understood by a person skilled in the art that the moisturizing agent may be any suitable synthetic or natural moisturizer that will assist in retaining moisture in the nail.

In a preferred embodiment the solution comprises from <1% to about 25% by weight of moisturizing agent, <1% to about 25% by weight of dispersant and <1% to about 60% by weight of water. The solution optionally may also include from <1% to about 25% solubilizing agent.

Examples of formulations of the solution of the present invention include but are not limited to the following examples:

### EXAMPLE 1

A solution, for topical application to a nail of a patient comprising between about 30% to about 85% of DMSO, or analogs thereof, by weight of solution, between about 0 to about 60% Water, by weight of solution, between about 0 to about 25% of at least one moisturizing agent, by weight of solution, and at least one medicine.

### EXAMPLE 2

A solution, for topical application to a nail of a patient comprising between about 30% to about 85% of DMSO, or analogs thereof, by weight of solution, between about 0 to about 30% ethanol, by weight of solution, between about 0 to about 25% propylene glycol, by weight of solution, between about 0 to about 60% water and at least one medicine.

### EXAMPLE 3

A solution, for topical application to a nail of a patient comprising between about 30% to about 85% of DMSO, or analogs thereof, by weight of solution, between about 0 to about 25% of at least one moisturizing agent, by weight of solution, between about 0 to about 25% propylene glycol, by weight of solution, between about 0 to about 60% water and at least one medicine.

### EXAMPLE 4

A solution, for topical application to a nail of a patient comprising between about 30% to about 85% of DMSO, or analogs thereof, by weight of solution, between about 0 to about 30% ethyl acetate, by weight of solution, between about 0 to about 30% butyl acetate, by weight of solution, between about 0 to about 30% isopropyl alcohol, by weight of solution, between about 0 to about 25% ethanol, by weight of solution, between about 0 to about 25% of at least one moisturizing agent, by weight of solution, between about 0 to about 60% water and at least one medicine.

### EXAMPLE 5

A solution, for topical application to a nail of a patient comprising between about 30% to about 85% of DMSO, or analogs thereof, by weight of solution, between about 0 to about 30% ethyl acetate, by weight of solution, between about 0 to about 30% isopropyl alcohol, by weight of solution, between about 0 to about 25% ethanol, by weight of solution, between about 0 to about 25% of at least one moisturizing agent, by weight of solution, between about 0 to about 60% water and at least one medicine.

### EXAMPLE 6

A solution, for topical application to a nail of a patient comprising between about 30% to about 85% of DMSO, or analogs thereof, by weight of solution, between about 0 to about 30% butyl acetate, by weight of solution, between about 0 to about 30% isopropyl alcohol, by weight of solution, between about 0 to about 25% ethanol, by weight of solution, between about 0 to about 25% of at least one moisturizing agent, by weight of solution, between about 0 to about 60% water and at least one medicine.

### EXAMPLE 7

A solution, for topical application to a nail of a patient comprising between about 30% to about 85% of DMSO, or analogs thereof, by weight of solution, between about 0 to about 30% ethyl acetate, by weight of solution, between about 0 to about 30% butyl acetate, by weight of solution, between about 0 to about 60% water and at least one medicine.

The present invention also provides for the use of the solution discussed above, for topical application of a medicine to a nail of a patient.

The present invention will now be illustrated by reference to the following examples which set forth advantageous embodiments. For example the carrier solution used in the following examples comprises 45% DMSO, or analogs thereof, 11% glycerin, 11% ethanol, 11% propylene glycol and water. However, variations to the components used within the solution and to the amounts can be made within the scope of the invention. It should be noted that these embodiments are illustrative and are not to be construed as restricting the invention in any way.

A percutaneous drug-enhancing formulation containing the absorption enhancer dimethylsulfoxide (DMSO) is the test carrier formulation in the following examples.

### MATERIALS AND METHODS

### Model Compounds

[¹⁴C]-Urea (specific activity 55 mCi/mmol, 99% purity), [7-¹⁴C]-salicylic acid (specific activity 55 mCi/mmol, 99% purity), and [³-H(G)]-ketoconazole (specific activity 5 Ci/mmol, 99% purity) were purchased from American Radio-labeled Chemicals, Inc. (ARC, St. Louis, Mo.).

### Human Finger Nail Plates

Nail plates were collected from adult human cadavers and stored in a closed container at 0° C. Before each experiment, nail samples were gently washed with normal saline to remove any contamination, then rehydrated by placing them for 3 h on a cloth wetted with normal saline.

### Preparation of Formulation

A formulation (Dimethaid Research Inc., Markham, Ontario, Canada) had previously been shown to enhance skin penetration formulation (test carrier formulation), as discussed in Hui X, et al., 1998, Pharm Res 15:1589-1595. The test formulation contains 45% DMSO, 11% glycerin, 11% ethanol, 11% propylene glycol and water, as discussed in Hewitt P G, et al., 1998, Pharm Res 15:988-992. Normal saline (ABBOTT Laboratories, North Chicago, Ill.) was the control. Concentrations (w/v) of [¹⁴C]-urea, [¹⁴C]-salicylic acid, and [³H]-ketoconazole were 0.002%, 0.068%, and 0.154% in the test carrier formulation, and 0.002%, 0.066%, and 0.098% in normal saline control, respectively. Measured pH values for test carrier and saline formulations were 6.8 and 5.7, respectively. pKₐ values for urea, ketoconazole, and salicylic acid were 0.1, 2.9 (and 6.5 as pKₐ2), and 2.9, respectively. Neither vehicle increased ionization or changed the solubility of these test chemicals in the vehicles. A 5 μL aliquot was removed from each vial and radioactivity was measured in a Packard Liquid Scintillation Counter (model 1500). A 5 μL aliquot carrier formulation contained 0.1 μCi/0.1 μg Eq. of [¹⁴C]-urea, or 0.1 μCi/3.4 μg Eq. of [¹⁴C]-salicylic acid, or 0.07 μCi/7.7 μg Eq. of [³H]-ketoconazole. For controls, a 5 μL aliquot saline formulation contained 0.1 μCi/0.1 μg Eq. of [¹⁴C]-urea, or 0.09 μCi/3.3 μg Eq. of [¹⁴ C]-salicylic acid, or 0.04 μCi/4.9 μg Eq. of [³H]-ketoconazole.

### Dosing and Surface Washing Procedures

A 5 μL dosing aliquot of each of the six test solutions was applied to the surface of a nail plate with a microsyringe twice daily, approximately 8 h apart for 7 days. Starting the second day, each morning before dosing, the surface of the nail was washed with cotton tips in a cycle, as follows: a dry tip, then a tip wetted with 50% skin cleansing liquid Ivory soap, then a tip wetted with distilled water, then another tip wetted with distilled water, then a final dry tip. This simulated a daily bathing. The washing samples from each cycle from each nail were pooled and collected by breaking off the cotton tip into scintillation glass vials. An aliquot of 5.0 mL methanol was added into each vial to extract test material. The radioactivity of each sample was measured in a liquid scintillation counter.

### Nail Incubation System

A Teflon® one-chamber diffusion cell (AMIE Systems) was used to hold each nail. To approximate physiological conditions, a small cotton ball wetted with 0.1 mL normal saline was placed in the chamber to serve as a “nail bed” and provide moisture for the nail plate. On day 5, 0.1 mL normal saline was injected through the inlet into the chamber to keep the cotton ball wet. A 1.5×1.5 cm aluminum sheet (0.24 mm thickness) with a hole in the center (1.0 cm diameter) was centered and glued to the top (dorsal) surface of the nail as a “frame” for the seal. The aluminum sheet frame flattens the curved nail and extends the surface area of the nail plate to fit the size of the top cover, so that potential leaking is prevented. The nail plate was placed on a ledge inside the receptor (1.0 cm in diameter and 0.5 cm high), sheet side up. The ventral (inner) surface of the nail was placed face down and rested on the wet cotton ball. Hydration of the nail plate and the supporting cotton bed was measured with a relative humidity/temperature meter (Corneometer CM 820; Courage & Khazaka, Cologne, Germany). Hydration was expressed digitally in arbitrary units (AU).

The rim of the cell base was covered with silicon gel and the top cover was set into the gel to prevent leaking. The metal holder was then tightly screwed into place.

The cells were placed on a platform in a large glass holding tank filled with saturated sodium phosphate solution. A digital relative humidity/temperature meter was used for monitoring room temperature, the chamber temperature, and humidity. The holding tank was then covered, thereby maintaining the cells at a constant humidity of 40%.

### Sampling Instrument and Procedure

The sampling instrument consisted of a drill (Dremel™ model 2850; Dremel Inc., Racine, Wis.), a drill press (Dremel™), an adjustable stage (Fisher, Pittsburgh, Pa.) with an adjustable holder, and a high-speed cutter (5/16 inch or 7.9 mm in diameter).

After completion of the incubation phase, the nail plate was removed from the diffusion cell. The aluminum sheet frame was peeled off and placed in a glass scintillation vial. The nail plate was transferred to a clean Teflon® diffusion cell for processing. The nail plate was inverted so that the ventral (nail bed) surface faced up and the dorsal (outer) dosed surface faced down. A top cover was tightly screwed into place. The cell was placed in the middle of the holds on the adjustable stage. The stage was pushed to its lowest position. The adjustable stage was then raised until the nail plate was just touching the tip of the cutter. The drill press was raised, and, without moving the Fisher stage, one piece of aluminum sheet (0.24 mm in thickness) was placed under the bottom of the diffusion cell to elevate it. The drill was turned on and the press was again pushed to its lowest position, removing a nail core sample of approximately the thickness of the aluminum sheet, 0.24 mm in depth and 7.9 mm in diameter, from the center of the ventral (nail bed) surface of the nail. The depth of the core thus removed by the drill was confirmed by measuring the difference in the thickness of nail before and after sampling with a digital micrometer (Sony M-30; Sony Magnescale Inc., Japan).

The drilling removed the nail sample as a powder. The powered nail samples were collected with a small paint brush and aluminum funnel into a glass scintillation vial and weighed. An aliquot of 5.0 mL Packard soluene-350 was added to the scintillation vial to dissolve the powder.

The upper part, the intermediate and dorsal layers of the center of the nail, including the area of application of the dose, was cut in the same diameter as the sampled area. The sample was then placed into a glass scintillation vial and dissolved by adding 5.0 mL Packard soluene-350. The rest of the nail was also dissolved in 5.0 mL Packard soluene-350. All samples were incubated at 40° C. for 48 h, followed by the addition of 10 mL scintillation cocktail (Hionic-fluor; Packard Instrument Company, Meriden, Conn.).

The radioactivity of each sample was counted by a liquid scintillation counter (Packard model 1500; Packard Instrument Company, Downer Grove, Ill.). ³H efficiency of the scintillation counter is equal to or larger than 60% and ¹⁴C efficiency is equal to or larger than 95%.

The amount of nail sample removed was also measured by the difference in weight of the nail plate before and after drilling and collecting the core of powder.

### Experimental Conditions

During the experiment, the holding tank temperature was 25±2° C. and relative humidity was 44±8%. Hydration (AU) of the dorsal nail surface was 8.5±2.4 for saline formulation and 11.2±3.6 for test carrier formulation. The supporting cotton bed hydration (AU) was 115.9±9.9 for saline formulation and 118.0±9.4 for test carrier formulation.

### EXAMPLE 1

### Characteristics of Nail Samples

Table 1 shows for each experiment the thickness of whole nail plate, the depth of the ventral surface core sample removed by the cutter, the percentage of the whole nail thickness, the total weight of the nail sample removed, and the actual weight of powered nail sample collected. The weight of the nail samples collected was consistent for all experiments.

TABLE 1

| | | NAIL CORE SAMPLED FROM | | | |
|---|---|---|---|---|---|
| | | THE VENTRAL (INNER) SURFACE | | | |
| | | CENTER OF THE NAIL PLATE* | | | |
| | Whole | Depth | | Total Core | Powder |
| | Nail | of | % Whole | Sample | Sample |
| | Thickness | Core | Nail | Removed | Collected |
| Test Number | (mm) | (mm) | Thickness | (mg) | (mg) |
| Urea | 0.65 | 0.25 | 39.52 | 16.4 | 5.2 |
| (Saline) | (0.09) | (0.03) | (8.05) | (4.3) | (0.8) |
| Urea | 0.71 | 0.27 | 37.97 | 17.6 | 6.4 |
| (test) | (0.07) | (0.03) | (2.69) | (4.3) | (1.3) |
| Ketoconazole | 0.68 | 0.28 | 41.88 | 14.3 | 6.7 |
| (saline) | (0.05) | (0.03) | (1.16) | (6.7) | (2.6) |
| Ketoconazole | 0.73 | 0.28 | 38.62 | 14.1 | 4.3 |
| (test) | (0.03) | (0.02) | (2.69) | (5.1) | (1.6) |
| Salicyclic acid | 0.77 | 0.25 | 32.62 | 12.1 | 6.0 |
| (saline) | (0.07) | (0.08) | (9.38) | (2.4) | (0.5) |
| Salicyclic acid | 0.6 | 0.21 | 35.03 | 23.4 | 4.7 |
| (test) | (0.12) | (0.06) | (6.45) | (8.3) | (0.8) |
| Average | 0.69 | 0.26 | 37.61 | 16.3 | 5.5 |
| | (0.09) | (0.05) | (6.20) | (6.2) | (1.6) |

*Nail sample, approximately 0.24 mm in depth and 1.9 mm in diameter, was drilled from the center of the ventral surface of the nail. The amount of nail sample removed was measured by difference in weight and depth of the drilled area before and after sampling. Each number represents mean ± SD of five tests.

### EXAMPLE 2

### Comparison of Chemical Penetration of Two Formulations

Table 2 shows weight normalized drug penetration (μg Eq. of ketoconazole, urea, and salicylic acid/g nail sample) into the combined dorsal and intermediate layers of the center of the nail plate with the test carrier formulation and saline formulation. Both descriptive and statistical analysis (student's t test, p<0.05) show a greater penetration of drug into the ventral (inner) layer of the nail plate with the test carrier than with saline control: ketoconazole, p=0.048; urea, p=0.039; salicylic acid, p=0.008.

TABLE 2

| | Radioactive Content | | |
|---|---|---|---|
| | (μg Eq./g Nail Sample) | | |
| Drug | Saline | Test Carrier | |
| Nail Layer | Formulation^{a} | Formulation^{a} | p Value |
| Ketoconazole | | | |
| Dorsal/intermediate center^{b} | 444.1 (121.0) | 289.2 (209.4) | 0.190 |
| Ventral/intermediate center^{c} | 34.0 (15.9) | 53.9 (10.6) | 0.048 |
| Urea | | | |
| Dorsal/intermediate center^{b} | 0.4 (0.3) | 0.8 (0.5) | 0.054 |
| Ventral/intermediate center^{c} | 0.2 (0.09) | 0.35 (0.15) | 0.039 |
| Salicyclic acid | | | |
| Dorsal/intermediate center^{b} | 71.6 (10.6) | 16.3 (4.2) | 0.000 |
| Ventral/intermediate center^{c} | 7.0 (1.1) | 10.2 (0.6) | 0.008 |

^{a}Each number represents mean ± SD of five tests.
^{b}7.9 mm diameter core sample cut from the dosed area in the center of the dorsal surface of the nail.
^{c}7.9 mm diameter core powdered nail sample removed by the drill from the ventral (inner) surface center of the nail, beneath the dosed area

### EXAMPLE 3

### Radioactive Content Recovery of [¹⁴C]-Salicylic Acid

During the experiment, a “pooling effect” was observed. The drying process of topically applied salicylic acid or urea in the carrier formulation was much slower when compared with saline controls. Thus, the determination of the distribution of radioactive content is of great interest. Table 3 shows the radioactivity recovery and mass balance for dosed salicylic acid in the two formulations. Note that after topical dosing in test carrier formulation, the dorsal, dosed surface of the nail remained visibly wet, with an apparent limited penetration of the nail plate, leading us to conclude that the surface wash contained most of the recovered drug. After topical dosing of urea in the test carrier, again some surface pooling was observed. In contrast, with each of these two chemicals the saline formulation dried within 60 min. There was no visible wetness observed with ketoconazole.

TABLE 3

| | Percent Applied Dose | |
|---|---|---|
| | | Test |
| Nail | Saline Formulation^{a} | Carrier Formulation^{a} |
| Dorsal/intermediate center^{b} | 47.10 (7.76) | 4.96 (2.13) |
| Ventral/intermediate center^{c} | 2.53 (0.51) | 7.04 (2.67) |
| Remainder | 14.20 (5.10) | 3.87 (1.26) |
| Subtotal | 63.83 (9.54) | 15.87 (3.76) |
| Surface Washing | 20.69 (5.97) | 70.86 (2.62) |
| Cotton supporting bed | 3.66 (2.02) | 2.23 (0.75) |
| Total | 88.18 (5.52) | 88.96 (2.29) |

^{a}Each number represents mean ± SD of five tests.
^{b}7.9 mm diameter core sample cut form the dosed area in the center of the dorsal surface of the nail.
^{c}7.9 mm diameter core powdered nail sample removed by the drill form the ventral (inner) surface center of the nail, beneath the dosed area.

Studies have previously shown that this percutaneous drug-enhancing formulation enhanced the permeability of human shin to diclofenac sodium, as discussed in Hewitt P G, et al., 1998, Pharm Res 15:988-992. With multiple dosing (four doses/day for 2 days) in vivo, human skin penetration of diclofenac in the carrier continually increased over the 48 h of study, as compared with a saline control. An in vivo human study, as discussed in Hui X, et al., 1998, Pharm Res 15:1589-1595 found that after a single topical application of radiolabeled diclofenac in that formulation on the human knee area, there was a continuous delivery of diclofenac from the carrier through the skin and into the urine, until the dosed site was washed at 24 h postdosing. In the present invention the efficacy of the carrier on the permeability of human nail plate to three radiolabeled chemicals, urea, salicylic acid, and ketoconazole was measured. Three chemicals were chosen as model compounds because they are frequently found in formulations. Salicylic acid and urea are often used topically for a variety of conditions, and ketoconazole is a widely used synthetic imidazole antifungal. However, it will be understood by a person skilled in the art that other chemicals may be chosen that are suitable for topical application. After topical application of each chemical twice daily for 7 days, the radioactivity content in the dorsal/top center dosed area, the intermediate center (beneath the dosed area), and ventral layer of the nail plate was determined. The results show that the carrier formulation increased radioactivity significantly in the ventral surface of the nail plate by approximately 50% compared with saline control (p<0.05).

There is a large difference in the mass balance ratios of salicylic acid between nail content and surface wash. With the saline formulation the top of the nail retains a large portion of the dosed drug after washing. The salicylic acid may interact and bond with the nail surface and thus be unavailable for absorption further into the nail. The test carrier formulation secures the salicylic acid and makes it available for further nail absorption.

The test carrier formulation enhanced penetration of radiolabeled urea, salicylic acid, and ketoconazole into the intermediate nail center as much as 50% higher than with saline controls (p<0.008-0.05) after 7 day repeated topical dosing.

All references referred to in the specification are herein incorporated by reference. While the embodiments discussed herein are directed to particular implementations of the invention, it will be apparent that variations and modifications may be effected thereto, by those of skill in the art, without departing from the scope of the invention.

1. A solution, for topical application to a nail of a patient, comprising
between about 30% to about 85% DMSO, or analogs thereof, by weight of the solution;
at least one medicine;
water; and
at least one of a moisturizing agent for assisting in the retention of moisture in the nail and preventing the nail from dehydrating, a dispersant for assisting the dispersion of the components in solution to provide a homogeneous solution when applied and when penetrating the nail, and a solubilizing agent for the medicine.

2. A solution according to claim 1 wherein the solution comprises between about 60% to about 70% DMSO, or analogs thereof, by weight of the solution.

3. A solution according to claim 1 wherein the solution comprises about 65% DMSO, or analogs thereof, by weight of the solution.

4. A carrier solution, for use with a medicine, for topical application to a nail of a patient, the solution comprising:
between about 30% to about 85% DMSO, or analogs thereof, by weight of the solution;
water; and
at least one of a moisturizing agent for assisting in the retention of moisture in the nail and preventing the nail from dehydrating, a dispersant for assisting the dispersion of the components in solution to provide a homogeneous solution when applied and when penetrating the nail, and a solubilizing agent for the medicine.

5. A solution according to claim 5 wherein the solution comprises between about 60% to about 70% DMSO, or analogs thereof, by weight of the solution.

6. A solution according to claim 5 wherein the solution comprises about 65% DMSO, or analogs thereof, by weight of the solution.
